(19) United States
(12) Patent Application Publication
(10) Pub. No.: US 2019/0212349 A1
(43) Pub. Date: Jul. 11, 2019

(54) INTEGRATED APPARATUS FOR DIAGNOSTIC ANALYSES

(71) Applicant: ALIFAX S.R.L., Polverara (IT)

(72) Inventor: Paolo Galiano, Padova (IT)

(73) Assignee: ALIFAX S.R.L., Polverara (IT)

(21) Appl. No.: 16/315,423

(22) PCT Filed: Jul. 4, 2017
(86) PCT No.: PCT/IB2017/054022

§ 371 Date: Jan. 4, 2019

(30) Foreign Application Priority Data
Jul. 4, 2016 (IT) 102016000069410

(51) Int. Cl. G01N 35/00; G01N 35/02; G01N 35/10; C12Q 1/18

(52) U.S. Cl. CPC G01N 35/0099; G01N 35/028; G01N 35/109; G01N 2035/042; C12Q 1/18; G01N 2035/00356; G01N 2035/00435; G01N 35/1004

(57) ABSTRACT

An integrated apparatus for diagnostic analyses includes a support structure; a first refrigerated container an analysis area in which microplates are positioned and having receptacles or wells for receiving a primary sample; a samples removal and delivery unit to remove a primary sample from test tubes and deliver it into the wells; a temperature control area containing the primary samples; and a robotic head configured to interact with said samples removal and delivery unit so as to transfer the primary samples taken from the test tubes to the temperature control area and configured to insert, into each of the wells, the sample where a bacterial growth has been identified, an antibiotic in liquid form according to a choice at the discretion of the operator directed as a function of the type of species identified.

### FIELD OF THE INVENTION

The present invention concerns an integrated apparatus for carrying out diagnostic analyses on a biological sample, native or taken from the patient. The invention is used to verify the presence of one or more bacteria in the sample, to classify or identify the type in order to select the appropriate antibiotics, useful for a possible therapy, which will then be analyzed together with the bacterium identified, in order to verify the effectiveness and, in any case, to provide an automatic flow of bacteriological analyses without any manual intervention by the operator, starting from taking the sample in the urine collection container, test tube, various container or other.

The biological sample to be analyzed, or primary biological sample, can be, for example, urine, or other sterile and non-sterile human biological liquids. The automation steps of the analytical procedures provide: bacterial growth in liquid eugonic broth, identification of the bacterial species by biochemical tests, automatic measurement of the McFarland 0.5 turbidity value and dispensation of liquid-phase antibiotics to provide the antibiogram suitable for the bacterial species and MIC, that is, the minimal inhibitory concentration for each single antibiotic selected.

### BACKGROUND OF THE INVENTION

Various techniques are known in the field of diagnostic analyses to check the presence of pathogenic organisms and microorganisms in a biological sample, to classify and/or identify their bacterial species and to identify a group of antibiotics able to initiate a targeted antibiotic therapy. This last operation is technically called antibiogram.

Known techniques for performing the antibiogram provide verification of the functionality of the antibiotics in suspensions of isolated bacteria, obtained after growth on solid media seeded on Petri dishes which require incubations of 12/24 hours per sample.

Known bacterial identification procedures provide analysis techniques, of the biochemical type, always starting from isolated colonies.

Especially for severe infections, the time needed to carry out the culture test, that is, the evaluation of bacterial growth, the identification and the execution of the antibiogram is long and this can pose dangers to the patient. It is therefore a common use of the medical class to administer, in advance to the patient, a broad spectrum antibiotic, without the support of diagnostic tests and solely on the basis of clinical suspicion, to allow the therapy to start immediately.

The indiscriminate use of such antibiotics induces so-called phenomena of drug-resistance. In fact, one disadvantage deriving from the use of such broad spectrum antibiotics consists, for example, in the fact that despite the fact that these drugs are initially effective in counteracting bacterial growth, it may happen that not only are they not able to completely eradicate all the bacterial colonies, but also that the surviving bacteria become resistant to the selected antibiotic through genetic mutations and subsequently proliferate, thus increasing the infection.

The scientific publication Barnes et al., Journal of Clinical Microbiology Vol. 12, No. 4, October 1980, “Clinical Evaluation of Automated Antibiotic Susceptibility Testing with the MS-2 System”, is known, which describes an automatic antibiotic sensitivity analysis starting from bacteria preliminarily isolated in Petri dishes, or disks. However, obtaining isolated bacteria requires that seeding has already been carried out, manually or automatically. Furthermore, Barnes et al. provides a manual visual adjustment by the operator of the desired value of McFarland turbidity, and uses pre-selected cartridges to carry out the antibiogram.

One solution to the disadvantages indicated above has been proposed in patent application WO-A-2006/021519, in the name of the present Applicant and relating to an integrated device for diagnostic analyses.

This solution, although effective, since it allows to obtain in a very short time an indication of the positivity of a sample and the selection of an effective family of antibiotics, has been shown to be open to improvement in terms of recognition and isolation of the type of germ or bacterium present in the positive sample.

In patent application WO-A-2010/097683, in the name of the present Applicant, an integrated device for diagnostic analyses is described able to offer a complete and automated type of bacteriological examination, in particular to effect bacterial growth and antibiogram, which allows on the one hand to obtain a fast and sufficiently reliable result and, on the other hand, to have confirmation of the results with classical methods, in a completely automated way, that is, reducing to a minimum the intervention of the operator, with obvious operational benefits starting with the taking of the initial sample.

This solution, although extremely effective, has been shown over time to be open to improvement in several aspects, for example from the point of view of transport and feeding of the test tubes, from the point of view of sampling and dispensing the sample from the test tubes, the sterilization process of the elements involved in or contributing to the sampling and dispensation of the sample and others.

Moreover, this device has shown itself to be open to improvement from the point of view of the layout and operating methodology of the various blocks or operating elements of which it consists, especially in relation to the possibility of perfecting the antibiogram, implementing it with other operating types of analysis, such as for example, the implementation of the Minimal Inhibitory Concentration or MIC.

Other limitations and disadvantages of conventional solutions and technologies will be clear to a person of skill after reading the remaining part of the present description with reference to the drawings and the description of the embodiments that follow, although it is clear that the description of the state of the art connected to the present description must not be considered an admission that what is described here is already known from the state of the prior art.

There is therefore a need to obtain an integrated apparatus for diagnostic analyses that can overcome at least one of the disadvantages of the state of the art.

One purpose of the present invention is therefore to provide an integrated apparatus for diagnostic analyses in which it is possible to perform, in a completely automated manner, tests to identify samples and the following synergic antibiogram, perfected by the implementation of the minimal inhibitory concentration or MIC.

Another purpose of the present invention is to obtain an integrated apparatus for diagnostic analyses which includes a disposition of the operating groups or elements of which it consists that is more effective and performing than known devices.

Another purpose of the present invention is to obtain an integrated apparatus for diagnostic analyses in which the automation and working capacity is considerably increased, especially as regards the transport and feed of the test tubes containing the primary samples.

Another purpose of the present invention is to obtain an integrated apparatus for diagnostic analyses that is efficient and fast from the point of view of taking and dispensing the sample, and which is extremely effective from the point of view of the sterilization of the elements involved in or contributing to the sampling and dispensation of the sample.

The Applicant has devised, tested and embodied the present invention to overcome the shortcomings of the state of the art and to obtain these and other purposes and advantages.

### SUMMARY OF THE INVENTION

The present invention is set forth and characterized in the independent claim, while the dependent claims describe other characteristics of the invention or variants to the main inventive idea.

In accordance with the above purposes and according to a first aspect of the invention, an integrated apparatus for diagnostic analyses comprises a support structure inside which are positioned a first refrigerated container to house at least one panel of antibiotics contained in ampoules or phials, reconstituted with liquid to allow them to be dispensed in a liquid phase, and to be tested according to a plurality of molecules which can be selected by an operator, also in concentrations, in order to carry out a modulatable antibiogram and MIC for each antibiotic chosen. There is an analysis area in which a plurality of microplates are positioned with a plurality of receptacles or wells in which a portion of a primary sample is inserted; a samples removal and delivery unit configured to remove a portion of a primary sample from respective test tubes and deliver it into the wells of the microplates, a temperature control area of the microplates containing the primary samples, and a robotic head configured to interact with the samples removal and delivery unit so as to transfer the primary samples taken from the test tubes in the microplates of the analysis area and to transfer the microplates to the temperature control area, and configured to insert, into each of the wells of the microplates, the sample where a bacterial growth has been identified, a portion of one of said antibiotics in liquid form according to a choice at the discretion of the clinical operator directed as a function of the type of species identified.

Advantageously, therefore, in the present integrated apparatus for diagnostic analyses it is possible to carry out the identification tests on the sample and following synergic antibiogram implemented by the realization of the minimal inhibition concentration of antibiotic, or MIC.

According to another aspect of the invention, the automatic antibiogram is carried out both from samples without identification, such as samples with sepsis, and also samples previously identified with chemical systems or other.

Furthermore, in the antibiogram performed automatically, the automatic detection of the McFarland 0.5 turbidity value is used.

The exclusivity of having antibiotics in liquid form differs from any other existing type, as other inventions propose a panel of antibiotics pre-deposited in microplates or cards with pre-set antibiotic panels. This advantage allows the operator to select antibiotics consistent with the species identified and to perform an antibiogram automatically in accordance with international standards and to perform said antibiogram from a McFarland 0.5 concentration, as required by international standards. Another exclusive aspect allows a “clinical” antibiogram to be performed on positive blood samples to test the efficacy of the antibiotic therapy given to the patient in cases of sepsis. These patients, defined as critical for their sepsis condition, are given specific antibiotics or a mixture of 3/5 antibiotics without waiting for the type of species of the infecting bacterium. Checking the antibiotic response, that is, the sensitive or resistant result, is of fundamental importance to the very life of the patient. The analysis time of a blood sample positive to the bacterial presence and antibiogram test can be obtained in 3-5 hours from the detection of the sample positive to bacterial hemo-culture by our method. The short time needed to verify whether an antibiotic administered is sensitive or resistant therefore also allows to verify the antibiotic therapy administered and, where appropriate, to change the antibiotic therapy initiated.

According to another aspect of the invention, the apparatus comprises a sample-carrying device, manually inserted and associated with the support structure and configured to allow a multiplicity of test tubes to be fed continuously to the apparatus and in particular to the samples removal and delivery unit.

In some embodiments, the sample-carrying device comprises an annular support associated with at least two return gear mechanisms of which at least one is motorized.

The apparatus can also comprise an interface associable with an automatic loading system of the test tubes to the apparatus. The interface can be used in combination with the manually inserted sample-carrying device.

The samples removal and delivery unit preferably comprises a plurality of needles associated with corresponding needle-carrying heads; said needle-carrying heads are configured to be selectively associated with the robotic head.

Advantageously, the samples removal and delivery unit can comprise a washing and/or sterilization unit of the needles.

In some embodiments, the apparatus comprises a magnetic-mechanical system configured to allow the selective mechanical and hydraulic connection of the robotic head to one of said needle-carrying heads.

According to another aspect of the invention, the apparatus comprises a device to read and identify the microplates.

The apparatus can comprise a second refrigerated container to temporarily park the samples able to be subsequently seeded on Petri dishes.

According to other aspects of the invention, the apparatus comprises a unit to read the microplates based on a light scattering technology, for application to culture tests, to residual antibiotic activity (RAA) tests and antibiogram.

Advantageously, in the present apparatus, it is therefore possible to house both the device for reading and identifying the microplates, such as a photometer, and also the unit for reading the microplates based on scattering technology.

The present invention therefore uses two different and distinct measuring technologies, that is, a photometer able to read the microplates, for the bacterial growth, antibiogram, and MIC and a laser scattering reading module for a clinical antibiogram, for blood cultures and other tests such as MDRO, or MRSA, ESBL or others.

These and other aspects, characteristics and advantages of the present disclosure will be better understood with reference to the following description, drawings and attached claims. The drawings, which are integrated and form part of the present description, show some forms of embodiment of the present invention, and together with the description, are intended to describe the principles of the disclosure.

The various aspects and characteristics described in the present description can be applied individually where possible. These individual aspects, for example aspects and characteristics described in the description or in the attached dependent claims, can be the object of divisional applications.

It is understood that any aspect or characteristic that is discovered, during the patenting process, to be already known, shall not be claimed and shall be the object of a disclaimer.

### BRIEF DESCRIPTION OF THE DRAWINGS

These and other characteristics of the present invention will become apparent from the following description of some embodiments, given as a non-restrictive example with reference to the attached drawings wherein:

FIG. 1 is a schematic view of the layout of the integrated apparatus for diagnostic analyses according to the present invention;

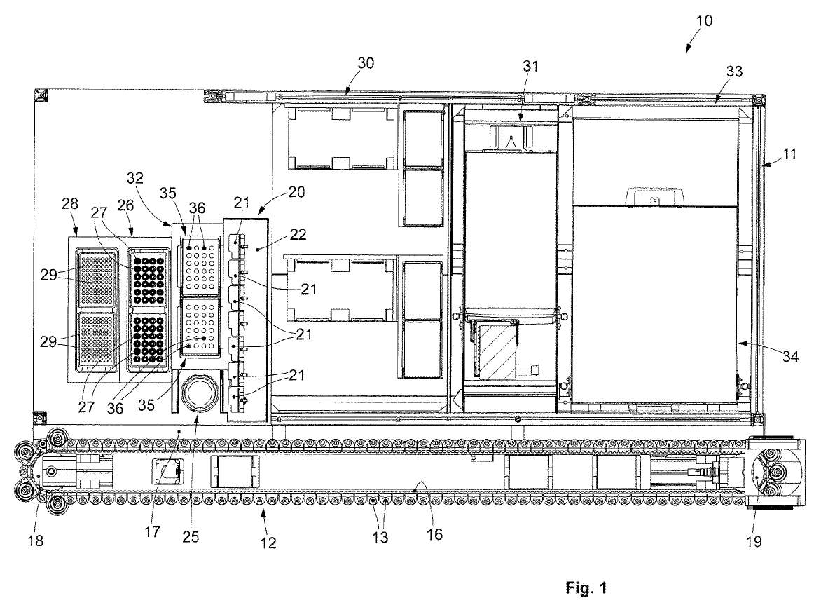

FIG. 2 is a front view of the present integrated apparatus for diagnostic analyses;

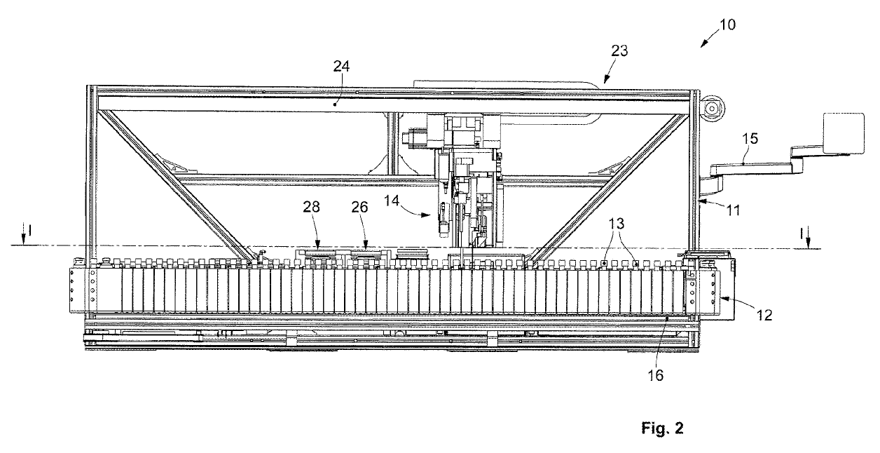

FIG. 3 is a side view of the present integrated apparatus for diagnostic analyses.

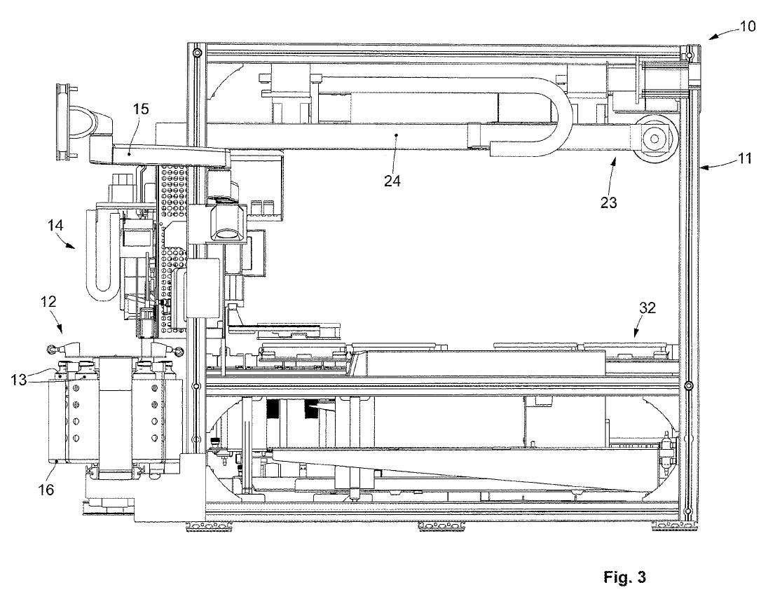

To facilitate comprehension, the same reference numbers have been used, where possible, to identify identical common elements in the drawings. It is understood that elements and characteristics of one embodiment can conveniently be incorporated into other embodiments without further clarifications.

### DETAILED DESCRIPTION OF SOME EMBODIMENTS

We shall now refer in detail to the various embodiments of the present invention, of which one or more examples are shown in the attached drawings. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description using the attached drawings. The present description can provide other embodiments and can be obtained or executed in various other ways. We must also clarify that the phraseology and terminology used here is for the purposes of description only, and cannot be considered as limitative.

With reference to the attached drawings, an integrated apparatus 10 for diagnostic analyses according to the invention comprises a support structure 11 to which a sample carrying device 12 to transport and feed samples is associated, for example a manually inserted sample carrying chain.

In the sample carrying device 12, a series of test tubes 13 are housed, inside each of which there is a pure biological sample, for example urine, or other sterile and non-sterile human biological liquids, or flasks of positive blood cultures.

The sample carrying device 12 comprises an annular support 16 that extends preferably over the whole front side 17 of the support structure 11.

The annular support 16 of the sample carrying device 12 comprises internally a toothing by which it engages in respective return gears 18 and 19, of which at least one is motorized.

The sample carrying device 12 has the task of allowing to load a large number of test tubes 13 and to carry the test tubes in correspondence with a samples removal and delivery unit 20.

The sample carrying device 12 is adaptable to various sample collection test tubes, both for urine and biological liquids as well as test tubes for blood cultures. A barcode identifies the type of sample loaded and consequently the appropriate workflow. If necessary, a positive blood culture sample will be dispensed directly into the container in which direct or clinical antibiogram tests will be performed in order to verify the sensitive or resistant response to the antibiotic therapy administered to the patient which takes, as described above, about 3 hours after inoculation into said clinical antibiogram test tubes.

The samples removal and delivery unit 20 comprises a plurality of needle-carrying heads 21, provided with corresponding actuators for lifting and lowering the needles, for example six needle-carrying heads as shown in the drawings.

The samples removal and delivery unit 20 also comprises a needle washing and/or sterilization unit 22, configured to dispense to a certain needle liquid disinfectant substances such as chlorine or suchlike, and to sterilize the needles, for example by heat. Each sample needs to be taken and dispensed, ensuring sterility of the needle, in order to avoid contamination in the dispensing. The structure provides the presence, for example, of 3/6 needles which, after the sample has been taken, are heated above 100° C. and subsequently washed with chlorine and then water. Each needle after this removal and sample dispensing process is inserted in a suitable decontamination chamber where through heating steps above 100° C., washing with chlorine and washing with water is sterilized for subsequent removal and dispensing processes.

Each of the needle-carrying heads 21 can also be connected to a pipe, for example, a flexible pipe cooperating with a pumping device for removing the liquid sample from the test tubes 13 and dispensing it in the various operating zones of the integrated apparatus 10.

Each needle-carrying head 21 can be mechanically and hydraulically engaged by a robotic head 14, for example by means of a magneto-mechanical system.

In the samples removal and delivery unit 20, the needles associated with each of the needle-carrying heads 21 are washed, sterilized by the unit 22 and parked for subsequent removal by the robotic head 14.

The robotic head 14 is associated with a movement unit 23 located, for example, in an upper zone of the support structure 11 and comprising guides 24 to allow the robotic head 14 to slide along at least two directions perpendicular to each other.

A data control and processing unit is associated with the support structure 11, for example by means of a support arm 15, from which it is possible to command and display the various operations performed by the integrated apparatus 10.

Next to the samples removal and delivery unit 20, there is provided an analysis area 32 in which one or more microplates 35 are positioned, comprising receptacles or wells 36 in which the primary samples are stored, which can be subjected to a rapid culture test.

In the analysis area 32, a given quantity of liquid is thus delivered, taken from the primary samples of the test tubes 13 in certain wells 36 of the microplate or microplates 35.

In a zone adjacent to the samples removal and delivery unit 20 and to the analysis area 32, a sensor 25 is provided, to verify the correct alignment of the needle of a certain needle-carrying head 21 engaged by the robotic head 14.

Next to the analysis area 32, a first refrigerated container 26 is advantageously positioned in which a plurality of phials 27 are housed in which antibiotics and/or other types of reagents are contained.

In the integrated apparatus 10 a second refrigerated container 28 is also provided, in which a series of receptacles 29, such as microwells, are made. A sample can be temporarily parked in the microwells, which will then be seeded on Petri dishes.

In a central zone of the integrated apparatus 10 a temperature control area 30 is provided, in which the microplates are positioned, keeping them at a constant temperature.

In a zone of the integrated apparatus advantageously adjacent to the temperature control area 30, a reading device 31 is provided, to read each of the wells 36 of the microplates 35, for example a photometer controlled by the control unit. The reading device 31 is useful for reading information on the biological sample contained in each microplate 35 and consequently of the patient from whom the biological sample was taken.

In the part of the integrated apparatus 10 located on the opposite side with respect to the analysis area 32, an interface 33 is provided with a possible automatic loading system of the test tubes on board the integrated apparatus, to be used alternatively or in combination with the manual load sample carrying device 12.

In the integrated apparatus 10 a reading group 34 is provided based on the light-scattering technology of the wells 36 of the microplates 35, for application to culture tests, tests of residual antibiotic activity or RAA (Residual Antimicrobial Activity), and antibiogram.

The present apparatus therefore advantageously uses two different and distinct measuring technologies, that is, a device 31 able to read microplates, for bacterial growth, antibiogram, and MIC, and a laser scattering reading unit 34 for a clinical antibiogram, for blood cultures and other tests such as MDRO, MRSA, ESBL and others.

The primary samples contained in the test tubes 13 are then inserted into the integrated apparatus 10 through two possible interfaces: a first manual interface, that is, through the sample carrying device 12, and/or through automatic loading devices that carry the test tubes 13 to the loading interface 33.

The robotic head 14 mechanically and hydraulically engages one of the needle-carrying heads 21 of the samples removal and delivery unit 20 through the magneto-mechanical system. The selected needle-carrying head will be provided with a sterilized needle by the washing and/or sterilization unit 22.

The needle-carrying head 21 with sterilized needle is used by the robotic head 14 to extract a certain quantity of primary sample from a particular test tube 13 which, thanks to the rotation of the annular support 16 of the sample carrying device 12, is taken into correspondence with the samples removal and delivery unit 20.

The removal of a certain quantity of primary sample from the test tube 13 is carried out by holing the stopper of the test tube 13 by the needle, or simply by immersing the needle into the primary sample of the test tube 13, if it is provided to take the test tube already opened into correspondence with the primary sample removal and delivery unit 20.

The quantity or portion of sample taken from the test tube 13 can be delivered partly into the receptacles 29 of the second refrigerated container 28 to allow possible seeding on Petri dishes, and partly into one or more wells 36 located on one or more microplates 35 of the analysis area 32.

The needle used to carry out the above removal operations of the primary sample from the test tube 13 and delivery into the second refrigerated container 28 and/or onto the analysis area 32 is returned by the robotic head 14 to the unit 22, in order to wash and/or sterilize it. Meanwhile, the robotic head 14 mechanically and hydraulically engages another needle-carrying head 21 with a previously sterilized needle, so the work cycle of the integrated apparatus is advantageously made continuous and without interruptions.

When a certain quantity of primary sample has been introduced into a certain number of wells 36 of one or more microplates 35 of the analysis area 32, the microplate 35 is moved by the robotic head 14 into the temperature control area 30 of the microplates.

Cyclically and always using the robotic head 14, each microplate 35 is taken to the microplate reader device 31 where it is possible to obtain and record the bacterial growth data for each specific well 36 of the microplate 35.

During the working cycle, therefore, the various readings relating to specific wells 36 of the microplate or microplates 35 are recorded and for each of the samples, over time, the curves of possible bacterial growth can be obtained.

The subsequent step in the work cycle is to identify bacteriological growth inside one of the wells 36 of the microplate 35. This step of bacterial growth can last for example from about 1 to 5 hours. As soon as bacterial growth is detected inside one or more wells 36 of the microplate 35, it means that said one or more wells 36 have positive samples.

The bacterium is then identified, which can be done by various methods and instruments, either internal or external to the integrated apparatus 10, for example mass spectrometers, or neural network algorithms or others.

The purpose of the step of identifying the bacterium is to establish the panel of antibiotics to be tested, therefore, as soon as the bacterium has been sufficiently identified and has reached the suitable concentration inside the corresponding well 36 of the microplate 35, it is re-suspended in a number of other wells 36 of the microplate 35 to be tested with different antibiotics delivered at different concentrations, that is, performing the antibiogram advantageously synergically. The antibiotics are always removed in perfect automation and by means of the robotic head 14 from the receptacles 29 of the second refrigerated container 28.

It is thus possible to measure the synergic effect of an antibiogram with different types of antibiotic that are distributed and act simultaneously.

By using the reader unit 34 based on light-scattering technology, it is also possible to carry out screening of bacteria resistant to one or more classes of antimicrobial agents, the so-called Multi-Drug Resistant Organisms (MDROs).

Inside the integrated apparatus 10, up to sixteen microplates 35 each containing twenty-four wells 36 can be provided, so that overall there can be 384 available positions to insert the sample.

As we said, after the identification of the bacterium, there follows the synergic antibiogram step, which can advantageously be implemented by the determination of the minimal inhibitory concentration or MIC, that is, the minimal amount of antibiotic to be administered to the patient.

The MIC is determined phenotypically according to the liquid dilutions technique. Let us suppose we have a panel of antibiotics to be tested comprising a certain number n of antibiotics contained in corresponding receptacles 29 of the second refrigerated container 28. Let us also suppose that m is the number of concentrations of antibiotic to be tested for each single antibiotic, therefore a sample in which bacterial growth has been detected and having a suitable concentration of the bacterium is delivered to n*m receptacles or wells 36 of a microplate 35. In each of these n*m wells 36 a certain quantity or amount of one of the n antibiotics is then added, such as to guarantee one of the m concentrations. This operation is done for each of the m concentrations of each of the n antibiotics.

In this way it is possible to carry out an effective synergic antibiogram, that is, to test the synergies of the different antibiotics.

The work cycle described above is concluded in perfect and complete automation, thanks to the robotic head 14, which moves appropriately from one zone of the integrated apparatus 10 to the other, thanks to the work capacity which is greatly increased by using a sample carrying device 12 and a possible additional automatic loading system for the test tubes, which is associated with the integrated apparatus 10 by the interface 33, and thanks to the samples removal and delivery unit 20, which comprises a plurality of needle-carrying heads 21 and an effective washing and/or sterilization unit 22 of said needles, so as to present a washed and/or sterilized needle continuously and for each sampling step.

In summary, therefore, the automated integrated device according to the invention advantageously provides to use antibiotics in liquid phase, which allows choice and discretion that can be customized by the end user, overcoming the use of predefined panels in quantity and type of antibiotic, offering the possibility of performing the antibiogram automatically.

The antibiogram can be performed automatically both from samples without prior identification, for example for patients with sepsis, and also from samples previously identified by various methods, chemical or otherwise.

The automatic antibiogram also uses automatic detection of McFarland 0.5 in the case of standard antibiogram.

The present apparatus also allows the use of direct clinical antibiogram for blood samples that are positive for bacterial growth in only three hours for the verification and confirmation of the antibiotic therapy administered to the patient, supplying the sensitive or resistant result.

As we have seen, the apparatus is provided with a sample loading chain or device that can be adapted to any type of test tube loaded, containing urine, biological fluid, or blood cultures.

The present apparatus is also provided with automatic means for sterilizing the sampling and dispensing needles, in order to obtain a workflow consistent with the sample capacity of the apparatus.

The present apparatus also advantageously uses two data acquisition technologies, namely photometry and laser scattering.

In the present apparatus, moreover, microplates of various sizes can be used, for example with 96, 192, 360 wells, pre-filled with eugonic broth, and suitable for bacterial growth, and for performing the antibiogram and MIC test.

It is clear that modifications and/or additions of parts may be made to the integrated apparatus for diagnostic analyses as described heretofore, without departing from the field and scope of the present invention.

It is also clear that, although the present invention has been described with reference to some specific examples, a person of skill in the art shall certainly be able to achieve many other equivalent forms of integrated apparatus for diagnostic analyses, having the characteristics as set forth in the claims and hence all coming within the field of protection defined thereby.

In the following claims, the sole purpose of the references in brackets is to facilitate reading: they must not be considered as restrictive factors with regard to the field of protection claimed in the specific claims.

1. An integrated apparatus for diagnostic analyses, comprising: a support structure inside which are positioned a first refrigerated container to house at least one panel of antibiotics contained in ampoules or phials, reconstituted with liquid to allow them to be dispensed in a liquid phase, and to be tested according to a plurality of molecules which can be selected by an operator and possibly also according to a plurality of concentrations, in order to carry out a modulatable antibiogram and MIC (Minimal Inhibitory Concentration) tests for each antibiotic chosen; an analysis area in which a plurality of microplates are positioned with a plurality of receptacles or wells in which a portion of a primary sample is inserted; a samples removal and delivery unit configured to remove a portion of a primary sample from respective test tubes and deliver it into the wells of said microplates; a temperature control area of the microplates containing the primary samples; and a robotic head configured to interact with said samples removal and delivery unit so as to transfer the primary samples taken from the test tubes in said microplates of the analysis area and to transfer said microplates to the temperature control area and configured to insert, into each of the wells of the microplates, the sample where a bacterial growth has been identified, a portion of one of said antibiotics in liquid form according to a choice at the discretion of the operator directed as a function of the type of species identified.

2. The apparatus as in claim 1, comprising implementation means of automatic antibiogram both from samples without identification, such as samples with sepsis, and also samples previously identified with chemical systems or other.

3. The apparatus as in claim 1, comprising automatic detection means of the McFarland 0.5 turbidity value.

4. The apparatus as in claim 1, comprising a sample-carrying device, manually inserted and associated with the support structure and configured to allow a multiplicity of test tubes to be fed continuously to the apparatus and in particular to the samples removal and delivery unit.

5. The apparatus as in claim 4, wherein said sample-carrying device comprises an annular support associated with at least two return gear mechanisms of which at least one is motorized.

6. The apparatus as in claim 1, comprising an interface associable with an automatic loading system of the test tubes to the apparatus.

7. The apparatus as in claim 1, wherein said samples removal and delivery unit comprises a plurality of needles associated with corresponding needle-carrying heads, said needle-carrying heads being configured to be selectively associated with said robotic head.

8. The apparatus as in claim 7, wherein said samples removal and delivery unit comprises a washing and/or sterilization unit of the needles.

9. The apparatus as in claim 7, comprising a magnetic-mechanical system configured to allow the selective mechanical and hydraulic connection of said robotic head to one of said needle-carrying heads.

10. The apparatus as in claim 1, comprising a device to read and identify the microplates.

11. The apparatus as in claim 1, comprising a second refrigerated container to temporarily park the samples able to be subsequently seeded on Petri dishes.

12. The apparatus as in claim 10, comprising a unit to read the microplates based on a light scattering technology, for application to culture tests, to residual antibiotic activity tests and antibiogram.
